(19) United States
(12) Patent Application Publication
(10) Pub. No.: US 2024/0199976 A1
(43) Pub. Date: Jun. 20, 2024

(54) LAUNDRY COMPOSITION

(71) Applicant: Conopco, Inc., d/b/a UNILEVER, Englewood Cliffs, NJ (US)

(72) Inventors: Karl BURGESS, Prenton, Wirral (GB); Alison CUMMINS, Ellesmere Port (GB); Joshua Chen Jae LOW, Moreton-in-Marsh (GB); Alastair Richard SANDERSON, Ellesmere Port (GB); Iain Murdoch STEVENSON, Bracknell (GB)

(73) Assignee: Conopco, Inc., d/b/a UNILEVER, Englewood Cliffs, NJ (US)

(21) Appl. No.: 18/286,727

(22) PCT Filed: Apr. 14, 2022
(86) PCT No.: PCT/EP2022/060024

§ 371 Date: Oct. 12, 2023

(30) Foreign Application Priority Data
Apr. 15, 2021 (EP) 21168527.6

(51) Int. Cl. C11B 9/00; C11D 3/50; C11D 7/26

(52) U.S. Cl. CPC C11B 9/0003; C11D 3/505; C11D 7/261

(57) ABSTRACT

A perfume particle composition comprising: a) at least 10 wt. % of a carrier material which comprises at least one ethoxylate unit and at least one carbon derived from carbon capture: b) perfume ingredients. 5

### FIELD OF THE INVENTION

The present invention relates to perfume particles comprising carbon from carbon capture.

### BACKGROUND OF THE INVENTION

Fragrance is an important aspect of the laundry process. Consumers often associate fragrance with cleanliness or simply enjoy the smell; accordingly, many laundry products comprise perfumes. However, the desired quantity of perfume varies from consumer to consumer. Consequently, perfume particles have been developed to allow consumers to tailor their perfume experience based on their person preferences.

Perfume particles may comprise ingredients comprising ethoxylate groups, such as alcohol ethoxylates and polyethylene glycol ingredients.

Fragrance performance is an essential feature for perfume particles. Many consumers judge the efficacy of the product based on perfume performance. Perfume performance may be judged on the product in the packaging, on wet fabrics, while drying, on dry fabrics, when folding and putting away, when wearing, or any combination of these touch points. Fragrance performance may be judged by quantity of fragrance, longevity or quality.

Stability is also an important feature of perfume particles. Instability is indicated by a change in the aesthetics, such as a colour change. Poor aesthetics can indicate poor stability. Equally aesthetics can be linked to the fragrance composition within a product.

There is a need to further improve perfume particles fragrance performance, aesthetics and/or stability.

In addition to the need for improved perfume particles, there is a growing need to address climate change, in particular greenhouse gases. There is a need to slow the rate at which carbon containing gases enter the atmosphere. In light of this, some consumers prefer so called ‘eco-friendly’ products which have a reduced impact on the environment. However often consumers associate ‘eco-friendly’ products reduced efficacy. Equally consumers can find it difficult to understand in tangible terms, the positive impact a product may have on the environment.

In view of the above, there remains a need for perfume particle compositions with a good environmental profile without compromising consumer satisfaction in terms of fragrance, stability and/or aesthetic performance.

### SUMMARY OF THE INVENTION

We have found that the perfume particle comprising at least 10 wt. % of a carrier material which comprises at least one ethoxylate unit and at least one carbon derived from carbon capture, provide an improved environmental profile while maintaining or improving consumer satisfaction. In particular, a difference in colour stability and fragrance profile is provided when an ingredient comprising at least one ethoxylate unit and at least one carbon derived from carbon capture are included in a perfume particle composition. The difference is colour stability provides improved stability in cold conditions. The difference in fragrance profile allows the consumer to identify a more environmentally friendly product and allows the producer the simplicity of continuing to use the same fragrance, but achieving a different fragrance profile. Without wishing to be bound by theory it is believed that improvements in the perfume particles are a consequence of the ingredients comprising carbon atoms from carbon capture.

In one aspect of the present invention is provided a perfume particle composition comprising:
a) at least 10 wt. % of a carrier material which comprises at least one ethoxylate unit and at least one carbon derived from carbon capture;
b) perfume ingredients.

The invention further relates to a method of preparing a perfume particle composition, wherein the method comprises the steps of:
i. Obtaining a carrier ingredient comprising at least one ethoxylate unit and at least one carbon derived from carbon capture;
ii. Melting said carrier material;
iii. Adding the perfume ingredients;
iv. Shaping and cooling the melt.

The invention additionally relates to a use of a perfume particle as described herein to reduce carbon emissions into the atmosphere

### DETAILED DESCRIPTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The term ‘virgin fossil fuels’ refers to fossil fuel sources (coal, crude oil, natural gas) which have not been used for any other purpose, i.e. has not been burnt for energy, or is not the waste gas from an industrial process.

The term ‘biomass’ refers to organic mass derived from plant materials and/or microorganisms (such as algae/microalgae/fungi/bacteria). Biomass includes, plant materials, agricultural residues/waste, forestry residues/waste, municipal waste provided this excludes fossil, yard waste, manufacturing waste, landfill waste, sewage sludge, paper and pulp etc. and the like.

The perfume particles described herein comprise carrier materials comprising at least one ethoxylate unit and at least one carbon derived from carbon capture. To obtain these carrier materials from carbon capture, carbon must be captured, separated (where required) and utilised or transformed into a carrier material for use in a perfume particles. The capture, separation and transformation may happen in one continuous process or may be separate steps which may be carried out at different locations.

### Carbon Capture and Separation

Carbon capture refers to the capture or sequestration of C1 carbon molecules (e.g. carbon monoxide, carbon dioxide, methane or methanol). By capturing the carbon molecules, they are removed from or prevented from entering the environment. Carbon sourced from carbon capture contrasts with carbon from virgin fossil fuels (crude oil, natural gas, etc.), in that captured carbon has already been used at least once; for example captured carbon may have been burned to produce energy and is captured to enable a second use of the carbon, whereas carbon from virgin fossil fuels have been extracted for that singular purpose. Captured carbon may equally be obtained from non-fossil fuel carbon emitters, such as biomass energy plants, brewery gases from fermentation (e.g. of wheat), burning of biomass fuels (e.g. vegetable oil, biogas or bio-ethanol). By capturing and utilising carbon, carbon can be used again, leading to less carbon in the atmosphere and reduced use of virgin fossil fuels. In other words by capturing carbon either already in the atmosphere or before it enters the atmosphere, the nett reliance on virgin fossil fuels to produce homecare products is reduced. The carbon captured may be in any physical state, preferably as a gas.

C1 carbon capture can be used to help reduce/prevent net release of CO₂ in the environment and thereby forms a valuable tool to address climate change. When the C1 carbons captured are derived from combusted fossil sources then the immediate CO₂ released can be reduced. When C1 carbons are derived directly from the atmosphere or from bio-sources there may even be a net immediate reduction in atmospheric CO₂ Carbon capture may be point source carbon capture or direct carbon capture. Direct carbon capture refers to capturing carbon from the air, where it is significantly diluted with other atmospheric gases. Point source carbon capture refers to the capture of carbon at the point of release into the atmosphere. Point source carbon capture may be implemented for example at steal works, fossil fuel or biomass energy plants, ammonia manufacturing facilities, cement factories, etc. These are examples of stationary point source carbon capture. Alternatively, the point source carbon capture may be mobile, for example attached to a vehicle and capturing the carbon in the exhaust gases. Point source carbon capture may be preferable due to the efficiency of capturing the carbon in a high concentration. Preferably, the carbon is captured from a point source. More preferably the carbon is captured from a fossil fuel based point source, i.e. carbon captured from an industry utilising fossil fuels.

There are various methods of capturing carbon from industrial processes, examples include:
Capturing carbon from flue gasses following combustion. This may be referred to as post combustion carbon capture. For example this may be implemented to capture carbon from the flue gasses at a fossil fuel power plant.
Capturing carbon pre-combustion. In these processes, fossil fuels are partially oxidized. Syngas comprising carbon monoxide, hydrogen and some carbon dioxide is produced. The carbon monoxide is reacted with water (steam) to produce carbon dioxide and hydrogen. The carbon dioxide can be separated, and the hydrogen used as fuel.
Oxy-fuel combustion, in which fuel is burned in oxygen rather than air. The flue gas consists mainly of carbon dioxide and water vapour. The water is separated and the carbon dioxide collected.

Once a source of carbon has been captured, the carbon molecules need to be isolated from the other chemicals with which they may be mixed. For example oxygen, water vapour, nitrogen etc. In some point source processes this step may not be required since a pure source of carbon is captured. Separation may involve biological separation, chemical separation, absorption, adsorption, gas separation membranes, diffusion, rectification or condensation or any combination thereof.

A common method of separation is absorption or carbon scrubbing with amines. Carbon dioxide is absorbed onto a metal-organic framework or through liquid amines, leaving a low carbon gas which can be released into the atmosphere. The carbon dioxide can be removed from the metal-organic framework or liquid amines, for example by using heat or pressure.

C1 carbon molecules sourced from carbon capture and suitably separated from other gases are available from many industrial sources. Suitable suppliers include Ineos.

Capturing carbon directly from the air may for example involve passing air over a solvent which physically or chemically binds the C1 molecules. Solvents include strongly alkaline hydroxides such as potassium or sodium hydroxide. For example air may be passed over a solution of potassium hydroxide to form a solution of potassium carbonate. The carbonate solution is purified and separated to provide a pure CO₂ gas. This method may also be employed in point source capture. An example of a direct air capture process is that employed by carbon engineering.

### Carbon Utilisation or Transformation

Once the C1 carbon molecules have been capture and separated, they can then be transformed into useful ingredients for use in a perfume particle.

Various methods may be used to transform the captured C1 molecules to useful components. The methods may involve chemical process or biological processes, such as microbial fermentation, preferably gas-fermentation.

Preferably the C1 molecules are transformed into:
i. Short chain (preferably C1-C5) intermediates such as methanol, ethanol, ethylene, ethylene oxide; or
ii. Hydrocarbon intermediates (preferably C6-C20) such as hydrocarbon chains: alkanes, alkenes, etc.

These can be converted further to make the components of surfactants, using well known chemistries e.g. chain growth reactions etc to: longer chain alkenes/olefins, alkanes, longer chain alcohols, aromatics and ethylene, ethylene oxide which is an excellent starter chemical for various ingredients. Preferably the C1 molecules are transformed into short chain intermediates, more preferably ethanol, ethylene or ethylene oxide.

### i. Short Chain Intermediates

One suitable example of transformation is a process in which a reactor converts carbon dioxide, water and electricity to methanol or ethanol and oxygen i.e. electrolysis. An example of this process is provided by Opus 12. Suitable processes are disclosed in WO21252535, WO17192787, WO20132064, WO20146402, WO19144135 and WO20112919.

An alternate suitable example of transformation is the conversion of carbon dioxide to ethanol using a catalyst of copper nanoparticles embedded in carbon spikes.

An alternate suitable example of transformation is the use of biological transformation which involves fermentation of the C₁ carbon by micro-organisms such as C₁-fixing bacteria to useful chemicals. This is alternatively known as gas fermentation, which is defined as the microbial conversion of gaseous substrates (e.g. CO, CO₂, and CH₄) to larger molecules.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl COA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. Preferably anaerobic bacteria such as those from the genus Clostridium are used to produce ethanol from carbon monoxide, carbon dioxide and hydrogen via the acetyl CoA biochemical pathway. There are a variety of microorganisms that can be used in a fermentation processes, particularly preferred are anaerobic bacteria such as Clostridium ljungdahlii strain PETC or ERI2, which can be used to produce ethanol.

Exemplary gas fermentation processes are, but not limited to, syngas fermentation and aerobic methane fermentation as described (B. Geinitz et. al. Gas Fermentation Expands the Scope of a Process Network for Material Conversion. Chemie Ingenieur Technik. Vol 92, Issue 11, p. 1665-1679.). The microbes with the ability to convert CO and CO₂ fall primarily into the group of anaerobic acetogenic bacteria or aerobic carboxydotrophic bacteria, those able to convert methane are methanotrophs, which are usually aerobic methanothrophic bacteria. In this sense the term ‘gas fermentation’ is used loosely and includes the aerobic or anaerobic microbial or enzymatic conversion of organic matter preferably by syngas fermentation and aerobic methane fermentation.

Gas-fermentation can include multi-stage fermentation, mixed fermentation, co-cultivation, mixotrophy and thermophilic production. Multi-stage fermentation can broaden the portfolio of products obtained together with higher end-product concentrations. Mixed fermentation may help some strains to detoxify the environment from a toxic compound or reduce the concentration of a certain product allowing for a more efficient conversion of the gas or increased product yield (e.g. by a second strain). Mixotrophy is the use of two or more carbon/electron sources simultaneously by some microorganisms, where for example both CO₂ and organic substrates such as sugars are utilized together. Thermophilic production (gas-fermentation at elevated temperatures by thermophilic strains, such as carboxydotrophic thermophiles) offers the advantages of reducing the risk of contamination. The gas-fermentation cultures may be defined or undefined, but preferably are in part or in the whole defined. Use of defined cultures offers the benefit of improved gas-fermentation end-product control.

Preferably the C1 molecules are transformed to short chain intermediates by gas fermentation. More preferably the C1 molecules are transformed to ethanol, ethylene or ethylene oxide by gas fermentation.

### ii. Hydrocarbon Intermediates

One suitable example is the Fischer-Tropsch process. Carbon dioxide and carbon monoxide can be chemically transformed to liquid hydrocarbons by the Fischer-Tropsch process, using hydrogen and a metal catalysis. Carbon dioxide feedstocks must first be converted to carbon monoxide by a reverse water gas shift reaction.

An alternate method for transformation into hydrocarbon intermediates solar photothermochemical alkane reverse combustion reactions. These are a one-step conversion of carbon dioxide and water into oxygen and hydrocarbons using a photothermochemical flow reactor.

Further examples of carbon capture technologies suitable to generate the ethanol stock for use in manufacturing ethoxy sub-units for use in the surfactants described herein are disclosed in WO 2007/117157, WO 2018/175481, WO 2019/157519 and WO 2018/231948.

### Percent Modern Carbon

The percentage modern carbon (pMC) level is based on measuring the level of radiocarbon (C14) which is generated in the upper atmosphere from where it diffuses, providing a general background level in the air. The level of C14, once captured (e.g. by biomass) decreases over time, in such a way that the amount of C14 is essentially depleted after 45,000 years. Hence the C14 level of fossil-based carbons, as used in the conventional petrochemical industry is virtually zero.

A pMC value of 100% biobased or biogenic carbon would indicate that 100% of the carbon came from plants or animal by-products (biomass) living in the natural environment (or as captured from the air) and a value of 0% would mean that all of the carbon was derived from petrochemicals, coal and other fossil sources. A value between 0-100% would indicate a mixture. The higher the value, the greater the proportion of naturally sourced components in the material, even though this may include carbon captured from the air.

The pMC level can be determined using the % Biobased Carbon Content ASTM D6866-20 Method B, using a National Institute of Standards and Technology (NIST) modern reference standard (SRM 4990C). Such measurements are known in the art are performed commercially, such as by Beta Analytic Inc. (USA). The technique to measure the C14 carbon level is known since decades and most known from carbon-dating archaeological organic findings.

The particular method used by Beta Analytic Inc., which is the preferred method to determine pMC includes the following:

Radiocarbon dating is performed by Accelerator Mass Spectrometry (AMS). The AMS measurement is done on graphite produced by hydrogen reduction of the CO₂ sample over a cobalt catalyst. The CO₂ is obtained from the combustion of the sample at 800° C.+ under a 100% oxygen atmosphere. The CO₂ is first dried with methanol/dry ice then collected in liquid nitrogen for the subsequent graphitization reaction. The identical reaction is performed on reference standards, internal QA samples, and backgrounds to ensure systematic chemistry. The pMC result is obtained by measuring sample C14/C13 relative to the C14/C13 in Oxalic Acid II (NIST-4990C) in one of Beta Analytic's multiple in-house particle accelerators using SNICS ion source. Quality assurance samples are measured along with the unknowns and reported separately in a “QA report”. The radiocarbon dating lab requires results for the QA samples to fall within expectations of the known values prior to accepting and reporting the results for any given sample. The AMS result is corrected for total fractionation using machine graphite d13C. The d13C reported for the sample is obtained by different ways depending upon the sample material. Solid organics are sub-sampled and converted to CO₂ with an elemental analyzer (EA). Water and carbonates are acidified in a gas bench to produce CO₂. Both the EA and the gas bench are connected directly to an isotope-ratio mass spectrometer (IRMS). The IRMS performs the separation and measurement of the CO₂ masses and calculation of the sample d13C.

In one embodiment, the carrier material comprising at least one ethoxylate unit and at least one carbon derived from carbon capture comprises carbons from point source carbon capture. These carrier materials preferably have a pMC of 0 to 10%.

In an alternate embodiment, the carrier material comprising at least one ethoxylate unit and at least one carbon derived from carbon capture comprises carbons from direct air capture. These carrier materials preferably have a pMC of 90 to 100%.

### Carrier Materials

By carrier is meant a solid material which provides the solid structure of the perfume particle. The compositions described herein preferably comprises at least 50 wt. % carrier materials, preferably 65 wt. %, more preferably 80 wt. % and most preferably at least 90 wt. % carrier materials, by weight of the composition. Preferably less than 98 wt. % carrier materials. This refers to the carrier material which comprises at least one ethoxylate unit and at least one carbon derived from carbon capture and any additional carrier materials.

Generally, carrier materials may be any material which disperses, dissolves, disintegrates or solubilises in water. The composition my comprise one carrier material or a combination of different carrier materials.

The perfume particles comprise at least 10 wt. % of a carrier material which comprises at least one ethoxylate unit and at least one carbon derived from carbon capture, more preferably at least 20 wt. %, even more preferably at least 50 wt. %, most preferably at least 60 wt. % and preferably less than 98 wt. %.

The carbon derived from carbon capture may be found anywhere within the chemical structure of the carrier material. Preferably the carbon derived from carbon capture forms part of an alkyl chain or an ethoxylate group, preferably an ethoxylate group. Preferably at least 50 wt. % of the carbon atoms in the carrier material are obtained from carbon capture, more preferably at least 70 wt. % and most preferably all of the carbon atoms are obtained from carbon capture. Preferably, less than 90 wt. %, preferably less than 10 wt. % of the carbon atoms within the carrier material are obtained directly from virgin fossil fuels.

### Carbon Located in Alkyl Chain of Carrier Material

Where the carbon derived from carbon capture is located in an alkyl chain, preferably on average at least 50 wt. % of the carbons in the alkyl chain are derived from carbon capture, more preferably at least 70 wt. %, most preferably all of the carbons in the alkyl chain are derived from carbon capture.

As described above, suitable carbon chains can be obtained from a Fischer-Tropsh reaction. The feedstock for the Fischer-Tropsch may be 100% carbon obtained from carbon capture, or may be a mixture of carbon from different sources. For example carbon gases from natural gas could be used, although this is not preferable. Preferably the alkyl chain comprises less than 10 wt. % carbon obtained directly from virgin fossil fuels more preferably the alky chain comprises no carbon obtained directly from virgin fossil fuels.

Alternatively the alkyl chain may be a combination of alkyl groups from carbon capture and alky groups from triglycerides, preferably triglycerides are obtained from plants, such as palm, rice, rice bran, sunflower, coconut, rapeseed, maze, soy, cottonseed, olive oil, etc.

### Carbon Located in Ethoxylate Group of Carrier Material

Where the carbon derived from carbon capture is located on an ethoxylate group, preferably on average at least 50 wt. % of the ethoxylate carbons in the molecule are derived from carbon capture, more preferably at least 70 wt. %, most preferably all the ethoxylate carbons in the molecule are derived from carbon capture. In a single ethoxylate monomer, one or both carbons may be carbons obtained from carbon capture, preferably both carbons are carbons obtained from carbon capture. Preferably, more than 10 wt. %, preferably more than 90 wt. % of the ethoxylate groups comprise carbon atoms obtained from carbon capture based sources. Alternate sources of carbon include plant based carbon, for example ethanol obtained from the fermentation of sugar and starch (i.e. ‘bio’ ethanol). The ethoxylate groups may comprise carbons from virgin fossil fuels, however this is not preferable. Preferably, less than 90 wt. %, preferably less than 10 wt. % of the ethoxylate groups comprise carbon atoms obtained directly from virgin fossil fuels.

To produce ethoxylates from carbon capture, first ethanol produced as outlined above is dehydrated to ethylene. This is a common industrial process. The ethylene is then oxidised to form ethylene oxide.

Depending on the desired carrier material, different routes are available.

If an alcohol ethoxylate is desired, the ethylene oxide can be reacted with a long chain fatty alcohol via a polymerisation type reaction. This process is commonly referred to as ethoxylation and gives rise to alcohol ethoxylates. Preferably the long chain fatty alcohol comprises carbon from carbon capture and/or from a plant source. More preferably the long chain fatty alcohol comprises only carbon from carbon capture and/or from a plant source. Most preferably and fatty alcohol comprises only carbon from carbon capture.

If a polyethylene glycol is desired, the ethylene oxide can be polymerised, for example in the presence of water and a catalyst to yield a polyethylene glycol chain.

Preferably all carbons within the carrier material ingredient molecule are derived from a plant source or carbon capture. Most preferably, all carbons are derived from carbon capture.

Preferred ethoxylated materials include: fatty acid ethoxylates, fatty amine ethoxylates, fatty alcohol ethoxylates, nonylphenol ethoxylates, alkyl phenol ethoxylate, amide ethoxylates, Sorbitan(ol) ester ethoxylates, glyceride ethoxylates (castor oil or hydrogenated castor oil ethoxylates) and mixtures thereof.

Preferably the carrier materials comprising at least one ethoxylate unit and at least one carbon derived from carbon capture is selected from alcohol ethoxylates, polyethylene glycols and combinations thereof.

### Alcohol Ethoxylates

Alcohol ethoxylates preferably have the general formula:

R¹O(R²O)ₓH
R¹=hydrophobic moiety.
R²=C₂H₄ or mixture of C₂H₄ and C₃H₆ units

x=4 to 120

R¹ preferably comprises 8 to 25 carbon atoms and mixtures thereof, more preferably 10 to 20 carbon atoms and mixtures thereof most preferably 12 to 18 carbon atoms and mixtures thereof. Preferably R¹ is selected from the group consisting of primary, secondary and branched chain saturated and/or unsaturated hydrocarbon groups comprising an alcohol, carboxy or phenolic group. Preferably R¹ is a natural or synthetic alcohol.

When the ingredient comprising at least one ethoxylate unit and at least one carbon derived from carbon capture is an alcohol ethoxylate, the carbon obtained from carbon capture may be located in the alky chain or the ethoxylate group. Preferably both the alkyl chain and ethoxylate comprise carbon obtained from carbon capture. Preferably R¹ comprises carbons from carbon capture.

R² preferably comprises at least 50% C₂H₄, more preferably 75% C₂H₄, most preferably R² is C₂H₄. Preferably R² comprises carbons from carbon capture.

x is preferably 8 to 90 and most preferably 30 to 90.

### Polyethylene Glycols

Polyethylene glycols (PEGs) have a general formula:

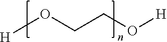

The weight average molecular weight of the PEG is preferably 2000 to 20000, more preferably 3000 to 15000, most preferably 4000 to 1200.

The PEG may solely comprise carbon from carbon capture or may comprise carbon from carbon capture in combination with carbon from other sources, as described above.

The perfume particles may comprise an additional carrier or a combination of additional carriers. The additional carriers materials may be selected from the group consisting of: non-carbon capture synthetic polymers (e g, polyethylene glycol, ethylene oxide/propylene oxide block copolymers, polyvinyl alcohol, polyvinyl acetate, and derivatives thereof), proteins (e.g., gelatin, albumin, casein), saccharides (e.g. dextrose, fructose, galactose, glucose, isoglucose, sucrose), polysaccharides (e.g., starch, xanthan gum, cellulose, or derivatives thereof), water-soluble or water dispersible fillers (e.g. sodium chloride, sodium sulfate, sodium carbonate/bicarbonate, zeolite, silica, clay), vegetable soap (e.g. coconut soap beads or palm soap), non-carbon capture ethoxylated non-ionic surfactants (having a formula R₁O(R₂O)ₓH, wherein R₁ preferably comprises 12 to 20 carbon atoms, R₂ is C₂H₄ or mixture of C₂H₄ and C₃H₆ units and x=8 to 120), urea and combinations thereof. By non-carbon capture it is meant that no carbon atoms are derived from carbon capture.

Examples of suitable carrier materials include: water soluble organic alkali metal salt, water soluble inorganic alkaline earth metal salt, water soluble organic alkaline earth metal salt, water soluble carbohydrate, water soluble silicate, water soluble urea, starch, xanthan gum, dextrose, clay, water insoluble silicate, citric acid carboxymethyl cellulose, fatty acid, fatty alcohol, glyceryl diester of hydrogenated tallow, glycerol, non-carbon capture polyvinyl alcohol, non-carbon capture non-ionic surfactants sold under the trade name Lutensol ex. BASF and combinations thereof.

Preferred additional carrier materials may be selected from the group consisting of non-carbon capture synthetic polymers (e g, polyethylene glycol, ethylene oxide/propylene oxide block copolymers, polyvinyl alcohol, polyvinyl acetate, and derivatives thereof), polysaccharides (e.g., starch, xanthan gum, cellulose, or derivatives thereof), saccharides (e.g, dextrose, fructose, galactose, glucose, isoglucose, sucrose), vegetable soap (e.g. coconut soap beads or palm soap), non-carbon capture ethoxylated non-ionic surfactants (having a formula R₁O(R₂O)ₓH, wherein R₁ preferably comprises 12 to 20 carbon atoms, R₂ is C₂H₄ or mixture of C₂H₄ and C₃H₆ units and x=8 to 120) and combinations thereof.

More preferably additional carriers are selected from starch, dextrose, coconut soap beads, palm soap and combinations thereof.

Saccharides are molecular compounds comprising carbon, hydrogen and oxygen. For the purposes of this invention a saccharide is defined as comprising one to ten monosaccharide units and mixtures thereof. In other words either a monosaccharide or an oligosaccharide or mixtures thereof. An oligosaccharide is a short saccharide polymer, typically considered in the art to comprise between two and ten monosaccharides units. It is preferred that a saccharide comprises 1 to 5 monosaccharide units, more preferably 1 to 4 monosaccharide units, most preferably the saccharide comprises monosaccharides, disaccharides or mixtures thereof. Disaccharides are the product of a reaction between two monosaccharides. They may be formed from two identical monosaccharides or two different monosaccharides. Examples of disaccharides include: sucrose, maltose, lactose. Monosaccharides are simple sugar units having the general formula (CH₂O)ₙ. Commonly n is 3, 5 or 6. According, monosaccharides can be classified by the number n, for example: trioses (e.g. glyceraldehyde), pentoses (e.g. ribose) and hexoses (e.g. fructose, glucose and galactose). Some monosaccharides may be substituted with additional functional groups, e.g. Glucosamine, others may have undergone deoxgenation and lost an oxygen atom e.g. deoxyribose. Therefore, the general chemical formulae can vary slightly depending on the monosaccharide.

Preferred monosaccharides for the present invention are hexose molecules (n=6). Hexose molecules all have the same molecular formula, however, have a different structural formula, i.e. are structural isomers. It is preferred that the hexose comprises a 6-membered ring, opposed to a 5 membered ring. Glucose and galactose have 6-membered rings. In a preferred embodiment the hexose monosaccharide is glucose. Glucose is a chiral molecule, having a mixture of D and L stereo isomers. Particularly preferably, the glucose of the present invention is the D isomer of glucose, also known as dextrose.

Preferably a saccharide material used in the present invention is anhydrous, i.e. free of any water. For example, dextrose monohydrate contains one molecule of water whereas anhydrous dextrose contains none.

Non-limiting examples of suitable saccharides for the present invention are: C*Dex ex Cargill, Treha ex Cargill, Anhydrous Dextrose ex Foodchem.

When a saccharide is used in the present invention, it may be preferable to include bitter material such as Bitrex ex Johnson Matthey Fine Chemicals, due to the sweetness of the saccharide.

### Perfume

The compositions of the present invention comprise perfume i.e. free oil perfume or non-confined perfumes. The compositions my preferably also comprise perfume microcapsules.

The compositions of the present invention may comprise one or more perfume compositions. The perfume compositions may be in the form of a mixture of free perfume compositions or a mixture of encapsulated and free oil perfume compositions.

Preferably the compositions of the present invention comprise 0.5 to 20 wt. % perfume ingredients, more preferably 1 to 15 wt. % perfume ingredients, most preferably 2 to 10 wt. % perfume ingredients. By perfume ingredients it is meant the combined free perfume and any encapsulated perfume.

Useful perfume components may include materials of both natural and synthetic origin. They include single compounds and mixtures. Specific examples of such components may be found in the current literature, e.g., in Fenaroli's Handbook of Flavor Ingredients, 1975, CRC Press; Synthetic Food Adjuncts, 1947 by M. B. Jacobs, edited by Van Nostrand; or Perfume and Flavor Chemicals by S. Arctander 1969, Montclair, N.J. (USA). These substances are well known to the person skilled in the art of perfuming, flavouring, and/or aromatizing consumer products.

Particularly preferred perfume components are blooming perfume components and substantive perfume components. Blooming perfume components are defined by a boiling point less than 250° C. and a LogP greater than 2.5. Substantive perfume components are defined by a boiling point greater than 250° C. and a LogP greater than 2.5. Preferably a perfume composition will comprise a mixture of blooming and substantive perfume components. The perfume composition may comprise other perfume components.

It is commonplace for a plurality of perfume components to be present in a free oil perfume composition. In the compositions for use in the present invention it is envisaged that there will be three or more, preferably four or more, more preferably five or more, most preferably six or more different perfume components. An upper limit of 300 perfume ingredients may be applied.

Free perfume may preferably be present in an amount from 0.01 to 20 wt. %, more preferably 0.1 to 15 wt. %, more preferably from 0.1 to 10 wt. %, even more preferably from 0.1 to 6.0 wt. %, most preferably from 0.5 to 6.0 wt. %, based on the total weight of the composition.

Preferably some of the perfume components are contained in a microcapsule. Suitable encapsulating materials may comprise, but are not limited to; aminoplasts, proteins, polyurethanes, polyacrylates, polymethacrylates, polysaccharides, polyamides, polyolefins, gums, silicones, lipids, modified cellulose, polyphosphate, polystyrene, polyesters or combinations thereof.

Perfume components contained in a microcapsule may comprise odiferous materials and/or pro-fragrance materials.

Particularly preferred perfume components are as described for free perfumes.

Encapsulated perfume may preferably be present in an amount from 0.01 to 20 wt. %, more preferably 0.1 to wt. 15%, more preferably from 0.1 to 10 wt. %, even more preferably from 0.1 to 6.0 wt. %, most preferably from 0.5 to 6.0 wt. %, based on the total weight of the composition.

### Cationic Polymers

The compositions of the present invention preferably comprise a cationic polymer. This refers to polymers having an overall positive charge. The compositions preferably comprise a cationic polymer at a level of from 0.1 to 5 wt. %, preferably from 0.1 to 4 wt. %, more preferably from 0.1 to 3 wt. %, even more preferably from 0.25 to 2.5 wt. %, most preferably from 0.25 to 1.5 wt. %.

The cationic polymer may be naturally derived or synthetic. Examples of suitable cationic polymers include: acrylate polymers, cationic amino resins, cationic urea resins, and cationic polysaccharides, including: cationic celluloses, cationic guars and cationic starches.

The cationic polymer of the present invention may be categorised as a polysaccharide-based cationic polymer or non-polysaccharide based cationic polymers. Polysaccharide-based cationic polymers:

Polysacchride based cationic polymers include cationic celluloses, cationic guars and cationic starches. Polysaccharides are polymers made up from monosaccharide monomers joined together by glycosidic bonds.

The cationic polysaccharide-based polymers present in the compositions of the invention have a modified polysaccharide backbone, modified in that additional chemical groups have been reacted with some of the free hydroxyl groups of the polysaccharide backbone to give an overall positive charge to the modified cellulosic monomer unit.

A preferred polysaccharide polymer is cationic cellulose. This refers to polymers having a cellulose backbone and an overall positive charge.

Cellulose is a polysaccharide with glucose as its monomer, specifically it is a straight chain polymer of D-glucopyranose units linked via beta-1,4 glycosidic bonds and is a linear, non-branched polymer.

The cationic cellulose-based polymers of the present invention have a modified cellulose backbone, modified in that additional chemical groups have been reacted with some of the free hydroxyl groups of the polysaccharide backbone to give an overall positive charge to the modified cellulose monomer unit.

A preferred class of cationic cellulose polymers suitable for this invention are those that have a cellulose backbone modified to incorporate a quaternary ammonium salt. Preferably the quaternary ammonium salt is linked to the cellulose backbone by a hydroxyethyl or hydroxypropyl group. Preferably the charged nitrogen of the quaternary ammonium salt has one or more alkyl group substituents.

Example cationic cellulose polymers are salts of hydroxyethyl cellulose reacted with trimethyl ammonium substituted epoxide, referred to in the field under the International Nomenclature for Cosmetic Ingredients as Polyquatemium 10 and is commercially available from the Amerchol Corporation, a subsidiary of The Dow Chemical Company, marketed as the Polymer LR, JR, and KG series of polymers. Other suitable types of cationic celluloses include the polymeric quaternary ammonium salts of hydroxyethyl cellulose reacted with lauryl dimethyl ammonium-substituted epoxide referred to in the field under the International Nomenclature for Cosmetic Ingredients as Polyquatemium 24. These materials are available from Amerchol Corporation marketed as Polymer LM-200.

Typical examples of preferred cationic cellulosic polymers include cocodimethylammonium hydroxypropyl oxyethyl cellulose, lauryldimethylammonium hydroxypropyl oxyethyl cellulose, stearyldimethylammonium hydroxypropyl oxyethyl cellulose, and stearyldimethylammonium hydroxyethyl cellulose; cellulose 2-hydroxyethyl 2-hydroxy 3-(trimethyl ammonio) propyl ether salt, polyquaternium-4, polyquaternium-10, polyquaternium-24 and polyquaternium-67 or mixtures thereof.

More preferably the cationic cellulosic polymer is a quaternised hydroxy ether cellulose cationic polymer. These are commonly known as polyquaternium-10. Suitable commercial cationic cellulosic polymer products for use according to the present invention are marketed by the Amerchol Corporation under the trade name UCARE.

The counterion of the cationic polymer is freely chosen from the halides: chloride, bromide, and iodide; or from hydroxide, phosphate, sulphate, hydrosulphate, ethyl sulphate, methyl sulphate, formate, and acetate.

### Non Polysaccharide-Based Cationic Polymers

A non-polysaccharide-based cationic polymer is comprised of structural units, these structural units may be non-ionic, cationic, anionic or mixtures thereof. The polymer may comprise non-cationic structural units, but the polymer must have a net cationic charge.

The cationic polymer may consists of only one type of structural unit, i.e., the polymer is a homopolymer. The cationic polymer may consists of two types of structural units, i.e., the polymer is a copolymer. The cationic polymer may consists of three types of structural units, i.e., the polymer is a terpolymer. The cationic polymer may comprises two or more types of structural units. The structural units may be described as first structural units, second structural units, third structural units, etc. The structural units, or monomers, may be incorporated in the cationic polymer in a random format or in a block format.

The cationic polymer may comprise a nonionic structural units derived from monomers selected from: (meth)acrylamide, vinyl formamide, N,N-dialkyl acrylamide, N,N-dialkylmethacrylamide, C1-C12 alkyl acrylate, C1-C12 hydroxyalkyl acrylate, polyalkylene glyol acrylate, C1-C12 alkyl methacrylate, C1-C12 hydroxyalkyl methacrylate, polyalkylene glycol methacrylate, vinyl acetate, vinyl alcohol, vinyl formamide, vinyl acetamide, vinyl alkyl ether, vinyl pyridine, vinyl pyrrolidone, vinyl imidazole, vinyl caprolactam, and mixtures thereof.

The cationic polymer may comprise a cationic structural units derived from monomers selected from: N,N-dialkylaminoalkyl methacrylate, N,N-dialkylaminoalkyl acrylate, N,N-dialkylaminoalkyl acrylamide, N,N-dialkylaminoalkylmethacrylamide, methacylamidoalkyl trialkylammonium salts, acrylamidoalkylltrialkylamminium salts, vinylamine, vinylimine, vinyl imidazole, quaternized vinyl imidazole, diallyl dialkyl ammonium salts, and mixtures thereof.

Preferably, the cationic monomer is selected from: diallyl dimethyl ammonium salts (DADMAS), N,N-dimethyl aminoethyl acrylate, N,N-dimethyl aminoethyl methacrylate (DMAM), [2-(methacryloylamino)ethyl]trl-methylammonium salts, N,N-dimethylaminopropyl acrylamide (DMAPA), N,N-dimethylaminopropyl methacrylamide (DMAPMA), acrylamidopropyl trimethyl ammonium salts (APTAS), methacrylamidopropyl trimethylammonium salts (MAPTAS), quaternized vinylimidazole (QVi), and mixtures thereof.

The cationic polymer may comprise a anionic structural units derived from monomers selected from: acrylic acid (AA), methacrylic acid, maleic acid, vinyl sulfonic acid, styrene sulfonic acid, acrylamidopropylmethane sulfonic acid (AMPS) and their salts, and mixtures thereof.

Some cationic polymers disclosed herein will require stabilisers i.e. materials which will exhibit a yield stress in the ancillary laundry composition of the present invention. Such stabilisers may be selected from: thread like structuring systems for example hydrogenated castor oil or trihydroxystearin e.g. Thixcin ex. Elementis Specialties, crosslinked polyacrylic acid for example Carbopol ex. Lubrizol and gums for example carrageenan.

Preferably the cationic polymer is selected from; cationic polysaccharides and acrylate polymers. More preferably the cationic polymer is a cationic polysaccharide. Even most preferably the cationic polymer is a cationic cellulose or guar. Most preferably the cationic polymer is a cellulose.

The molecular weight of the cationic polymer is preferably greater than 20 000 g/mol, more preferably greater than 25 000 g/mol. The molecular weight is preferably less than 2 000 000 g/mol, more preferably less than 1 000 000 g/mol.

### Optional Ingredients

The compositions of the present invention may contain further optional laundry ingredients. Such ingredients include colourants, preservatives, pH buffering agents, perfume carriers, hydrotropes, polyelectrolytes, anti-shrinking agents, anti-oxidants, anti-corrosion agents, drape imparting agents, anti-static agents, ironing aids, antifoams, colorants, pearlisers and/or opacifiers, natural oils/extracts, processing aids, e.g. electrolytes, hygiene agents, e.g. anti-bacterials and antifungals, thickeners, low levels of cationic surfactants such as quaternary ammonium compounds and skin benefit agents.

### Form of Composition

In one embodiment of the present invention is provided a method of preparing a perfume particle composition, wherein the method comprises the steps of:
i. Obtaining a carrier ingredient comprising at least one ethoxylate unit and at least one carbon derived from carbon capture;
ii. Melting said carrier material;
iii. Adding the perfume ingredients;
iv. Shaping and cooling the melt.

The perfume particles may be in any solid form, for example: powder, pellet, tablet, prill, pastille or extrudate. Preferably the composition in the form of a pastille or extrudate. Pastilles can, for example, be produced using ROTOFORMER Granulation Systems ex. Sandvick Materials.

The perfume particle compositions of the present invention may be formed from a melt. The solid composition can for example, be formed into particles by: Pastillation e.g. using a ROTOFORMER ex Sandvick Materials, extrusion, prilling, by using moulds, casting the melt and cutting to size or spraying the melt.

An example manufacturing process may involve melting the carrier material (including the carrier material comprising at least one ethoxylate unit and at least one carbon derived from carbon capture) at a temperature above the melting point of that carrier material, preferably at least 2° C. above the melting point of the carrier material, more preferably at least 5° C. above the melting point of the carrier material. Where more than one carrier materials are used, the melting point is considered to the highest of the melting points of the individual materials. Once melted, perfume and other ingredients may be mixed into the compositions. This is followed by a process in which the melt in cooled and shaped, e.g. extrusion or pastillation.

The perfume particle compositions of the present invention are preferably homogeneously structured. By homogeneous, it is meant that there is a continuous phase throughout the solid product. There is not a core and shell type structure. Any particles present such as perfume microcapsules will be distributed within the continuous phase. The continuous phase is provided predominately by the carrier materials. The perfume particle compositions may be any shape or size suitable for dissolution in the laundry process. Preferably, each individual particle of the solid composition has a mass of between 0.95 mg to 5 grams, more preferably 0.01 to 1 gram and most preferably 0.02 to 0.5 grams. Preferably each individual particle has a maximum linear dimension in any direction of 10 mm, more preferably 1-8 mm and most preferably a maximum linear dimension of 4-6 mm. The shape of the particles may be selected for example from spherical, hemispherical, compressed hemispherical, lentil shaped, oblong, or planar shapes such as petals. A preferred shape for the particles is hemispherical, i.e. a dome shaped wherein the height of the dome is less than the radius of the base. When the particles are compressed hemispherical, it is preferred that diameter of the substantially flat base provides the maximum linear dimension and the height of the particle is 1-5 mm, more preferably 2-3 mm. the dimensions of the particles of the present invention can be measured using Calipers.

Preferably the perfume particles are packaged in a container. When the container is plastic, preferably the container comprises recycled plastic, in particular PCR. “post-consumer resin (PCR)” typically means plastic that has been collected via established consumer recycling streams, sorted, washed and reprocessed, for example into pellets.

### Use of the Perfume Particles

In one aspect of the present invention is provided a use of perfume particles as described herein to reduce carbon emissions into the atmosphere. This is achieved by re-using carbon which is already in the atmosphere or which will be emitted into the atmosphere (e.g. from industry) rather than using carbon from virgin fossil fuels. The perfume particles as described herein can contribute to slowing the rate of carbon entering the atmosphere. In other words carbon derived from carbon capture can be used in a perfume particle to reduce carbon emissions in the atmosphere. This is achieved by re-using carbon which has been or will be emitted into the atmosphere rather than using virgin petrochemicals.

Additionally, the use of an ingredient comprising at least one ethoxylate unit and at least one carbon derived from carbon capture provides the consumer with a tangible eco marker in the product. Accordingly, in one aspect of the present invention is provided a use of an ingredient comprising at least one ethoxylate unit and at least one carbon derived from carbon capture as a tangible eco marker in a perfume particle composition. The tangible eco marks the change in carbon providence for the consumer. This may be a change in the smell of the product. In other words carbon derived from carbon capture may be used to change the fragrance of a perfume particle, thereby providing the consumer with a tangible marker and a reason to believe.

### EXAMPLES

The following ingredients are illustrative of carrier materials comprising at least one ethoxylate unit and at least one carbon derived from carbon capture.

TABLE 1
Alcohol ethoxylate

| | Ethoxylate (80 EO) | Alkyl (C16/18) |
|---|---|---|
| Comparative A | Virgin fossil fuel | Virgin fossil fuel |
| Example 1 | Virgin fossil fuel | Carbon Capture |
| Example 2 | Carbon Capture | Palm oil |
| Example 3 | Carbon Capture | Carbon Capture |

TABLE 2
Polyethylene glycol (molecular weight 8000)

| | Ethylene oxide |
|---|---|
| Comparative B | 100% Virgin fossil fuel |
| Example 4 | 30% Virgin fossil fuel, 70% carbon capture |
| Example 5 | 50% bioethanol, 50% carbon capture |
| Example 6 | 100% Carbon Capture |

The following compositions are perfume particle compositions according to the present invention:

TABLE 3
Perfume particles

wt. % active

| Ingredient | 7 | 8 |
|---|---|---|
| Alcohol ethoxylate - Example 3 | 90 | — |
| PEG - Example 4 | — | 90 |
| Blue dye ⁶ | — | 0.01 |
| Free perfume | 10 | 6 |
| Perfume microcapsules | — | 4 |

### Product Assessment

TABLE 4
Particle composition

wt. % inclusion

| Ingredient | A | 1 |
|---|---|---|
| PEG 8000¹ | 65 | — |
| PEG 8000² | — | 65 |
| Dextrose | 26.5 | 26.5 |
| Fragrance oil | 6.5 | 6.5 |
| Perfume microcapsule | 2 | 2 |
| Water | To 100 | To 100 |

PEG 8000¹—Polyethylene glycol have an average molecular weight of 8000 and carbon derived from petrochemical sources.

PEG 8000²—Polyethylene glycol have an average molecular weight of 8000 and carbon derived from carbon capture sources.

The particles were prepared by the following process. the PEG 8000 was heated to ˜65° C. The dextrose was added with stirring, followed by the fragrance oil and the microcapsules. The particles were formed by pipetting onto a flat surface.

To test the colour stability the perfume particles were stored at 5° C. Colour measurements were taken at 0 weeks to provide a baseline, colour change was assessed after 1 week. Colour was assessed by the ΔE value, comparing the aged sample to the 0 weeks sample. ΔE was calculated using the CIELAB colour space, wherein each colour has an L*, a* and b* value and

ΔE*_{ab}=√(L*₂-L*₁)+(a*₂-a*₁)+(b*₂-b*₁)

The colour assessment was performed using an X-rite VS450 colour spectrometer.

ΔE is the difference between initial and week 1 colour measurements.

TABLE 5
Colour stability measurements

| | Initial | 1 week | ΔE |
|---|---|---|---|
| A | 14.6 | 21.57 | 9.1 |
| 1 | 15.97 | 17.90 | 2.26 |

The perfume particles comprising PEG 8000 comprising carbon derived from carbon capture demonstrated less of a colour change than the PEG comprising carbon from petrochemical sources. Perfume particles 1 have improved colour stability at cold temperatures.

1. A perfume particle composition comprising:
a) at least 10 wt. % of a carrier material which comprises at least one ethoxylate unit and at least one carbon derived from carbon capture; and
b) perfume ingredients.

2. The perfume particle according to claim 1, wherein at least 50 wt. % of the carbon atoms in the ingredient a) are obtained from carbon capture.

3. The perfume particle according to claim 1, wherein less than 90 wt. %, of the carbon atoms in ingredient a) are obtained directly from virgin fossil fuel sources.

4. The perfume particle according to claim 1, wherein the carbon derived from carbon capture forms part of an alkyl chain or an ethoxylate group.

5. The perfume particle according to claim 1, wherein ingredient a) is selected from alcohol ethoxylates, polyethylene glycols and combinations thereof.

6. The perfume particle according to claim 1, wherein ingredient a) is an alcohol ethoxylate.

7. The perfume particle according to claim 1, wherein ingredient a) is a polyethylene alcohol having a molecular weight of 2,000 to 20,000.

8. The perfume particle according to claim 1, wherein all carbons within the ingredient a) are derived from carbon capture or a combination of carbon capture and plant source.

9. The perfume particle according to claim 1, wherein the carbon obtained from carbon capture is obtain from point source carbon capture.

10. The perfume particle according to claim 1, wherein the perfume particle comprises an additional carrier material, selected from: non-carbon capture synthetic polymers, proteins, saccharides, polysaccharides, water-soluble or water dispersible fillers, vegetable soap, non-carbon capture ethoxylated non-ionic surfactants, urea and combinations thereof.

11. The perfume particle according to claim 1, wherein the perfume ingredients comprise free perfume.

12. The perfume particle according to claim 1, wherein the perfume ingredients comprise encapsulated perfume.

13. A method of preparing a perfume particle composition, wherein the method comprises the steps of:
i. obtaining a carrier ingredient comprising at least one ethoxylate unit and at least one carbon derived from carbon capture;
ii. melting said carrier material to produce a melt;
iii. adding the perfume ingredients; and
iv. shaping and cooling the melt.

14. A method of preparing the perfume particle composition of claims 1, wherein the method comprises the steps of:
i. obtaining a carrier ingredient comprising at least one ethoxylate unit and at least one carbon derived from carbon capture;
ii. melting said carrier material;
iii. adding the perfume ingredients; and
iv. shaping and cooling the melt.

15. The method according to claim 14 wherein the method of preparing the perfume particle reduces atmospheric carbon emissions.
